(19) United States
(12) Patent Application Publication
(10) Pub. No.: US 2023/0157613 A1
(43) Pub. Date: May 25, 2023

(54) ELECTROCARDIOGRAPHIC DETECTION DEVICE FOR VEHICLE

(71) Applicant: KABUSHIKI KAISHA TOKAI-RIKA-DENKI-SEISAKUSHO, Niwa-gun, Aichi (JP)

(72) Inventors: Masaki KUNUGITA, Niwa-gun, Aichi (JP); Yusuke YAMANAKA, Niwa-gun, Aichi (JP); Atsushi HARADA, Niwa-gun, Aichi (JP)

(21) Appl. No.: 17/913,012

(22) PCT Filed: Mar. 15, 2021
(86) PCT No.: PCT/JP2021/010264

§ 371 Date: Sep. 20, 2022

(30) Foreign Application Priority Data
Mar. 25, 2020 (JP) 2020-055102

(51) Int. Cl. A61B 5/318; A61B 5/00; A61B 5/26

(52) U.S. Cl. CPC A61B 5/318; A61B 5/6893; A61B 5/26

(57) ABSTRACT

An electrocardiographic detection device for a vehicle includes: a steering wheel electrode provided at a lower layer of a covering material that covers a surface of a steering wheel; a steering wheel electrode provided at a lower layer of the covering material of the steering wheel and an insulating material of a predetermined thickness; a waveform generator detecting a differential voltage between the steering wheel electrode and a ground region; a waveform generator detecting a differential voltage between the steering wheel electrode and a ground region; and a signal processing section that generates an electrocardiographic signal on the basis of the differential voltage detected at the waveform generator and the differential voltage detected at the waveform generator.

### TECHNICAL FIELD

The present invention relates to an electrocardiographic detection device for a vehicle that detects an electrocardiographic waveform of a vehicle occupant.

### BACKGROUND ART

There is known a technique in which electrodes are provided at a steering wheel and/or a vehicle seat, and the electrocardiographic waveform of a vehicle occupant is detected.

Japanese Patent Application Laid-Open (JP-A) No. 2013-212311 discloses the invention of an electrocardiographic detection device for a vehicle having a steering wheel electrode that is disposed at the steering wheel of a vehicle and contacts the skin of a vehicle occupant and detects the potential of the body of the vehicle occupant, and a first capacitive coupling type electrode and a second capacitive coupling type electrode disposed at the backrest of a vehicle seat and detecting the potential of the body of the vehicle occupant without contacting the skin of the vehicle occupant, wherein the electrocardiographic waveform of the vehicle occupant is measured on the basis of the difference between the potential difference between the potential at the steering wheel electrode and the potential at the first capacitive coupling type electrode, and the potential difference between the potential at the steering wheel electrode and the potential at the second capacitive coupling type electrode.

### SUMMARY OF INVENTION

### Technical Problem

However, in the technique disclosed in JP-A No. 2013-212311, a grasped portion that is covered by a hand (body) by being grasped by the vehicle occupant, and a non-grasped portion that is not grasped by the vehicle occupant and is not covered by the body, arise at the steering wheel electrode. Of the steering wheel electrode, the non-grasped portion generates noise due to vibration of the vehicle, and there is the problem that, due to this noise being superposed on the electrocardiographic signal, it is difficult to detect the electrocardiographic signal.

FIG. 8 is an explanatory drawing that illustrates the path of generation and entry of noise at the electrocardiographic detection device for a vehicle. As illustrated in FIG. 8, in a structure such as that disclosed in JP-A No. 2013-212311, the following two types of noise that are caused by vibration of the vehicle body can be mentioned. (In FIG. 8, the arrows indicate the direction of the flow of current (noise).) One is common mode noise 130 that enters from capacitor CN2 formed by the charges generated at a body 12 side due to vibration, and flows via a seat electrode 122, which is the first capacitive coupling type electrode or the second capacitive coupling type electrode, to a ground region (GND). Another one is normal mode noise 132 that enters from a non-grasped portion 118 of a steering wheel electrode 116 that functions as a capacitor CN1 due to vibration, and flows via the seat electrode 122 to GND. At the time of detecting an electrocardiographic signal by the steering wheel electrode 116, the latter normal mode noise 132 is particularly problematic.

The normal mode noise 132 is current that is generated accompanying vibration of the electrode at the non-grasped portion 118 of the steering wheel electrode 116, and flows via the body 12 to GND, and flows also via a buffer circuit 140 to GND. The potential difference between GND and the potential at the steering wheel electrode is outputted from the output end of the buffer circuit 140. Therefore, there is the problem that accurate detection of an electrocardiographic signal is difficult due to the effects of the normal mode noise 132 that flows via the body 12 to GND.

The present disclosure provides an electrocardiographic detection device for a vehicle in which the S/N ratio of an electrocardiographic signal is high due to noise caused by vibration being suppressed.

### Solution to Problem

A first aspect of the present disclosure is an electrocardiographic detection device for a vehicle, comprising: a first electrode portion provided at a lower layer of a covering material that covers a surface of a steering wheel, a second electrode portion provided at a lower layer of the covering material of the steering wheel and an insulating material of a predetermined thickness; a first detecting portion detecting a differential voltage between the first electrode portion and a ground region; a second detecting portion detecting a differential voltage between the second electrode portion and the ground region; and a signal processing section that generates an electrocardiographic signal based on the differential voltage detected at the first detecting portion and the differential voltage detected at the second detecting portion.

In accordance with the first aspect, an electrocardiographic signal of a high S/N ratio and in which noise due to vibration is suppressed can be obtained by the processing of outputting the difference between the electrocardiographic signal detected at the first electrode portion and the signal detected at the second electrode portion and in which the normal mode noise 132 is seen markedly, or the like.

A second aspect of the present disclosure is an electrocardiographic detection device for a vehicle, comprising: a first electrode portion provided at a lower layer of a covering material that covers a surface of a steering wheel; a second electrode portion provided at a lower layer of the covering material of the steering wheel and an insulating material of a predetermined thickness; a third electrode portion provided at a seat in which a vehicle occupant sits; a first detecting portion detecting a differential voltage between the first electrode portion and a ground region; a second detecting portion detecting a differential voltage between the second electrode portion and a ground region; a third detecting portion detecting a differential voltage between the third electrode portion and a ground region; and a signal processing section that generates an electrocardiographic signal based on the differential voltage detected at the first detecting portion, the differential voltage detected at the second detecting portion, and the differential voltage detected at the third detecting portion.

In accordance with the second aspect, an electrocardiographic signal of a high S/N ratio and in which noise due to vibration is suppressed can be obtained by the processing of outputting the difference between the electrocardiographic signal detected at the first electrode portion, the signal detected at the second electrode portion and in which the normal mode noise 132 is seen markedly, and the signal detected at the third electrode portion and in which the common mode noise 130 is seen markedly, or the like.

Further, the first electrode portion and the second electrode portion respectively may be disposed so as to be symmetrical as seen from a radial direction of the steering wheel.

Further, as seen from the radial direction of the steering wheel, the first electrode portion and the second electrode portion respectively are adjacent to one another, respective shapes thereof are substantially congruent, and the first electrode portion and the second electrode portion may have a geometrically equivalent relationship in which respective surface areas thereof are substantially the same.

Further, the first electrode portion and the second electrode portion respectively may be disposed parallel as seen from a radial direction of the steering wheel.

Further, the first electrode portion and the second electrode portion may be disposed in forms of dots as seen from a radial direction of the steering wheel.

### BRIEF DESCRIPTION OF DRAWINGS

FIG. 1 is a drawing illustrating the schematic structure of an electrocardiographic detection device for a vehicle relating to a first embodiment.

FIG. 2A is a schematic drawing illustrating an example of an arranged pattern of steering wheel electrodes.

FIG. 2B is a schematic drawing illustrating another example of an arranged pattern of the steering wheel electrodes.

FIG. 3A is an enlarged drawing of the arranged pattern of the steering wheel electrodes illustrated in FIG. 2.

FIG. 3B is a cross-sectional view at line A-A of FIG. 3A.

FIG. 4A is a schematic drawing illustrating an example of an electrocardiographic signal that is detected at the steering wheel electrode and outputted from a waveform generator.

FIG. 4B is a schematic drawing illustrating an example of an electrocardiographic signal that is detected at the steering wheel electrode, which is separated from the surface of a steering wheel by an insulating material, and is outputted from a waveform generator.

FIG. 4C is a schematic drawing illustrating an example of an electrocardiographic signal that is outputted from a signal processing section.

FIG. 5A is a schematic drawing illustrating a modified example of the arranged pattern of the steering wheel electrodes.

FIG. 5B is a schematic drawing illustrating yet another example of a modified example of the arranged pattern of the steering wheel electrodes.

FIG. 6 is a drawing illustrating the schematic structure of an electrocardiographic detection device for a vehicle relating to a second embodiment.

FIG. 7A is a schematic drawing illustrating an example of an electrocardiographic signal that is detected at the steering wheel electrode and outputted from the waveform generator.

FIG. 7B is a schematic drawing illustrating an example of an electrocardiographic signal that is detected at the steering wheel electrode, which is separated from the surface of the steering wheel by an insulating material, and is outputted from a waveform generator.

FIG. 7C is a schematic drawing illustrating an example of an electrocardiographic signal that is detected at a seat electrode and outputted from a waveform generator.

FIG. 7D is a schematic drawing illustrating an example of an electrocardiographic signal that is outputted from a signal processing section.

FIG. 8 is an explanatory drawing illustrating a path of generation and entry of electrocardiographic noise at an electrocardiographic detection device for a vehicle.

### DESCRIPTION OF EMBODIMENTS

Examples of electrocardiographic detection devices for a vehicle relating to embodiments of the present disclosure are described in detail hereinafter with reference to the drawings.

### First Embodiment

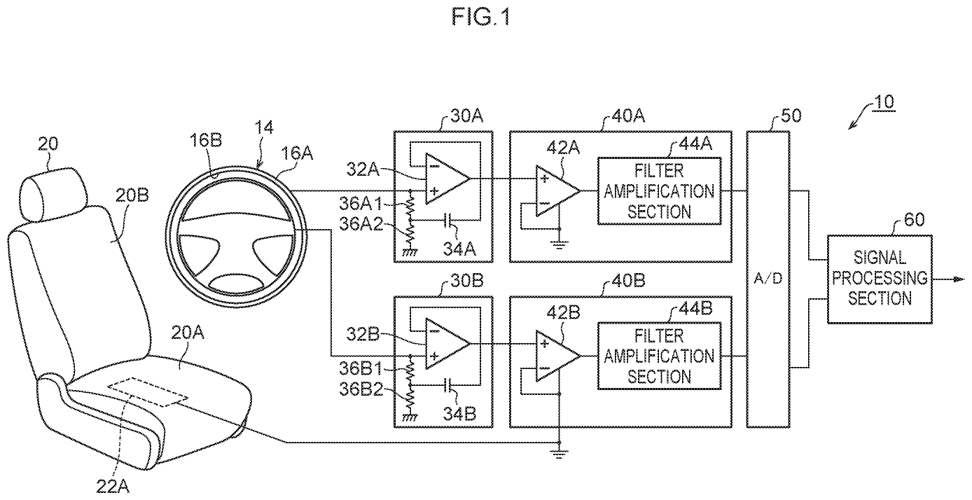

First, an electrocardiographic detection device for a vehicle relating to a first embodiment is described. FIG. 1 is a drawing illustrating the schematic structure of an electrocardiographic detection device 10 for a vehicle relating to the first embodiment.

The electrocardiographic detection device 10 for a vehicle relating to the present embodiment has steering wheel electrodes 16A, 16B, a seat electrode 22A, buffer circuits 30A, 30B, waveform generators 40A, 40B, an A/D converter 50, and a signal processing section 60.

The steering wheel electrodes 16A, 16B and the seat electrode 22A are provided as electrodes that are provided at positions at which a vehicle occupant can contact them.

The steering wheel electrodes 16A, 16B are provided along the entire peripheral direction region of a steering wheel 14 that is for carrying out steering operation of the vehicle. When the vehicle occupant grasps the steering wheel 14, the hands of the vehicle occupant contact the steering wheel electrodes 16A, 16B, and capacitive coupling arises between the hands of the vehicle occupant and the steering wheel electrodes 16A, 16B, and electrostatic capacitive coupling type capacitors are formed. Further, the steering wheel electrodes 16A, 16B are electrically connected to the waveform generators 40A, 40B via the buffer circuits 30A, 30B. The waveform generators 40A, 40B detect the ion current changes (alternating current), which accompany the electrical activity of the heartbeat of the heart, from the steering wheel electrodes 16A, 16B as current signals (electrocardiographic signals). As will be described later, the steering wheel electrode 16A is an electrode that detects mainly an electrocardiographic signal, and the steering wheel electrode 16B is an electrode that detects mainly noise that enters the electrocardiographic signal.

The seat electrode 22A is provided at a seat cushion 20A of a vehicle seat 20, which is further toward the vehicle lower side than the position of the heart of the vehicle occupant seated in the vehicle seat 20, and is covered by a seat cover (not illustrated). Due to the vehicle occupant sitting on the vehicle seat 20, the seat electrode 22A contacts the buttocks of the vehicle occupant via the clothing of the vehicle occupant and the seat cover, and forms an electrostatic capacitive coupling type capacitor that is capacitively coupled with the vehicle occupant. The seat electrode 22A is grounded together with the inverting input terminal (−) of a differential amplifier 42B that is described later, so that the static electricity generated by the clothes of the vehicle occupant seated in the vehicle seat 20 and the like flows to GND. The seat electrode 22A may be disposed at a seatback 20B of the vehicle seat 20 provided that it is at the lower side of the position of the heart of the vehicle occupant.

The buffer circuit 30A is provided between the steering wheel electrode 16A and the waveform generator 40A, and outputs the signal from the steering wheel electrode 16A to the waveform generator 40A as a buffer. The buffer circuit 30A is structured by a positive feedback circuit called a bootstrap circuit that includes an operational amplifier 32A, resistors 36A1, 36A2, and a capacitor 34A. More specifically, the steering wheel electrode 16A is electrically connected to the non-inverting input terminal (+) of the operational amplifier 32A, and is grounded via the resistors 36A1, 36A2. The resistors 36A1, 36A2 structure a type of voltage divider. The signal from the steering wheel electrode 16A, which is divided in accordance with the resistance values of the resistors 36A1, 36A2, is inputted via the capacitor 34A to the inverting input terminal (−) of the operational amplifier 32A.

The waveform generator 40A is structured to include a differential amplifier 42A that serves as a detecting portion, and a filter amplification section 44A, and generates an electrocardiographic waveform of the vehicle occupant on the basis of the current signal inputted from the steering wheel electrode 16A.

Due to the non-inverting input terminal (+) of the differential amplifier 42A being connected to the steering wheel electrode 16A via the buffer circuit 30A, and the inverting input terminal (−) being grounded, the differential voltage (differential signal) between the signal inputted to the non-inverting input terminal (+) and the potential of GND is detected, and is outputted to the filter amplification section 44A. Specifically, the differential amplifier 42A amplifies, by a predetermined factor, the difference between the input signal from the steering wheel electrode 16A and the potential of GND, and outputs a signal.

The filter amplification section 44A amplifies the output signal of the differential amplifier 42A, and carries out the processing of converting the resultant signal into a signal of a frequency of a predetermined range by using a predetermined filter, or the like, and outputs the results of processing via the A/D converter 50 to the signal processing section 60. A filter such as, for example, a high pass filter, a low pass filter, a bandpass filter or the like can be used appropriately as the predetermined filter. The signal that is outputted by the filter amplification section 44A is converted from an analog signal into a digital signal by the A/D converter 50, and is inputted to the signal processing section 60.

The buffer circuit 30B is provided between the steering wheel electrode 16B and the waveform generator 40B, and outputs the signal from the steering wheel electrode 16B to the waveform generator 40B as a buffer. In the same way as the buffer circuit 30A, the buffer circuit 30B is structured by a positive feedback circuit that includes an operational amplifier 32B, resistors 36B1, 36B2, and a capacitor 34B.

The waveform generator 40B is structured to include the differential amplifier 42B that serves as a detecting portion, and a filter amplification section 44B, and, on the basis of the current signal inputted from the steering wheel electrode 16B, generates an electrocardiographic waveform of the vehicle occupant that includes noise much more markedly than the signal generated by the waveform generator 40A.

Due to the non-inverting input terminal (+) of the differential amplifier 42B being connected to the steering wheel electrode 16B via the buffer circuit 30B, and the inverting input terminal (−) being grounded together with the seat electrode 22A, the differential voltage (differential signal) between the signal inputted to the non-inverting input terminal (+) and the signal inputted to the inverting input terminal (−) is detected, and is outputted to the filter amplification section 44B. Specifically, the differential amplifier 42B amplifies, by a predetermined factor, the difference between the input signal from the steering wheel electrode 16B and GND, and outputs a signal.

The filter amplification section 44B amplifies the output signal of the differential amplifier 42B, and, in the same way as the above-described filter amplification section 44A, carries out the processing of converting the resultant signal into a signal of a frequency of a predetermined range by using a predetermined filter, or the like, and outputs the results of processing via the A/D converter 50 to the signal processing section 60.

The signal processing section 60 is structured by a computer including a CPU (Central Processing Unit), a ROM (Read Only Memory), a RAM (Random Access Memory), and a non-volatile memory such as a flash memory or the like. The CPU executes signal processing by executing a program that is stored in advance in the memory.

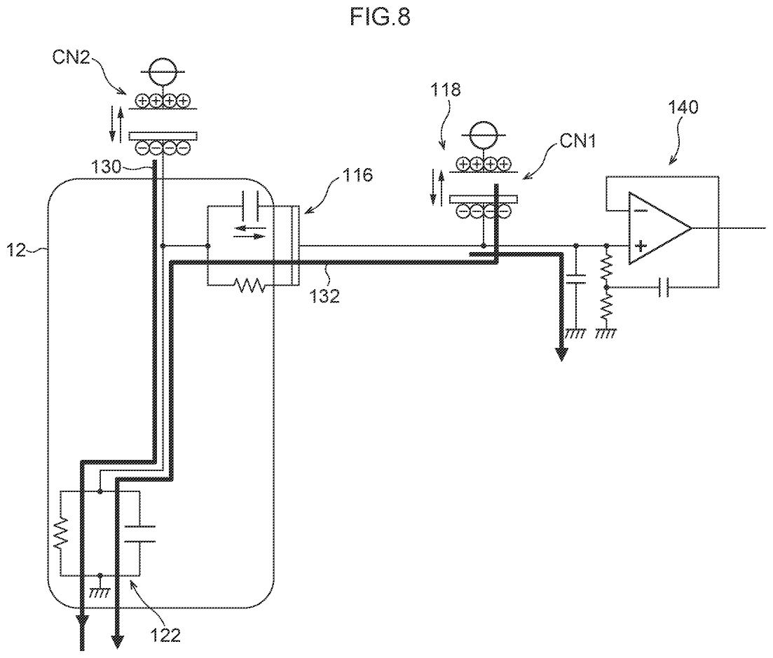

In the present embodiment, the processing of outputting the difference between a signal, in which the waveform of the electrocardiographic signal outputted from the waveform generator 40A is marked, and a signal, which markedly includes the normal mode noise 132 such as illustrated in FIG. 8 in the electrocardiographic signal outputted from the waveform generator 40B, or the like, is carried out. Due to this processing, the component relating to the normal mode noise 132 that is due to vibration is removed from the electrocardiographic signal outputted from the waveform generator 40A, and an electrocardiographic signal having a high S/N ratio is outputted.

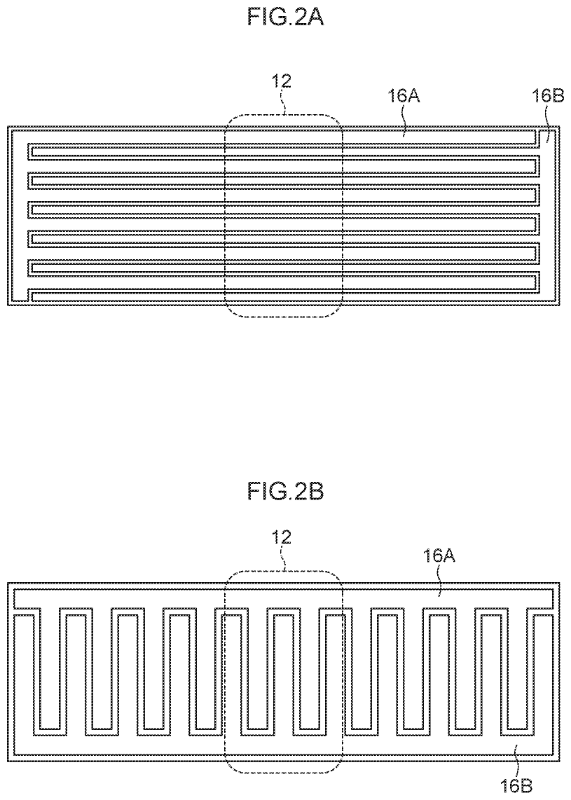

FIG. 2A is a schematic drawing illustrating an example of the arranged pattern of the steering wheel electrodes 16A, 16B, and FIG. 2B is a schematic drawing illustrating another example of the arranged pattern of the steering wheel electrodes 16A, 16B. The steering wheel electrodes 16A, 16B illustrated in FIG. 2A are respectively disposed parallel to the peripheral direction of the steering wheel 14. The steering wheel electrodes 16A, 16B illustrated in FIG. 2B are respectively disposed orthogonal to the peripheral direction of the steering wheel 14. In both cases of FIG. 2A and FIG. 2B, the aforementioned first electrode portion and the aforementioned second electrode portion are respectively disposed parallel and present an arranged pattern that is a striped pattern, as seen from the radial direction of the steering wheel 14.

As described above, the steering wheel electrodes 16A, 16B are provided over the entire peripheral direction region of the steering wheel 14, and the steering wheel electrode 16A and the steering wheel electrode 16B respectively are disposed so as to be symmetrical as seen from the radial direction of the steering wheel 14. More specifically, the steering wheel electrodes 16A, 16B respectively are adjacent to one another, and the respective shapes thereof are substantially congruent, and the steering wheel electrodes 16A, 16B have a geometrically equivalent relationship in which the respective surface areas thereof are substantially the same. Accordingly, as illustrated in both FIG. 2A and FIG. 2B, the respective surface areas of the steering wheel electrode 16A and the steering wheel electrode 16B at a region where the steering wheel 14 is grasped by a hand (the body 12) of the vehicle occupant are substantially the same. Further, the arranged pattern of the steering wheel electrodes 16A, 16B is such that, the more narrow the respective pitches of the steering wheel electrodes 16A, 16B, the greater the correlation with noise.

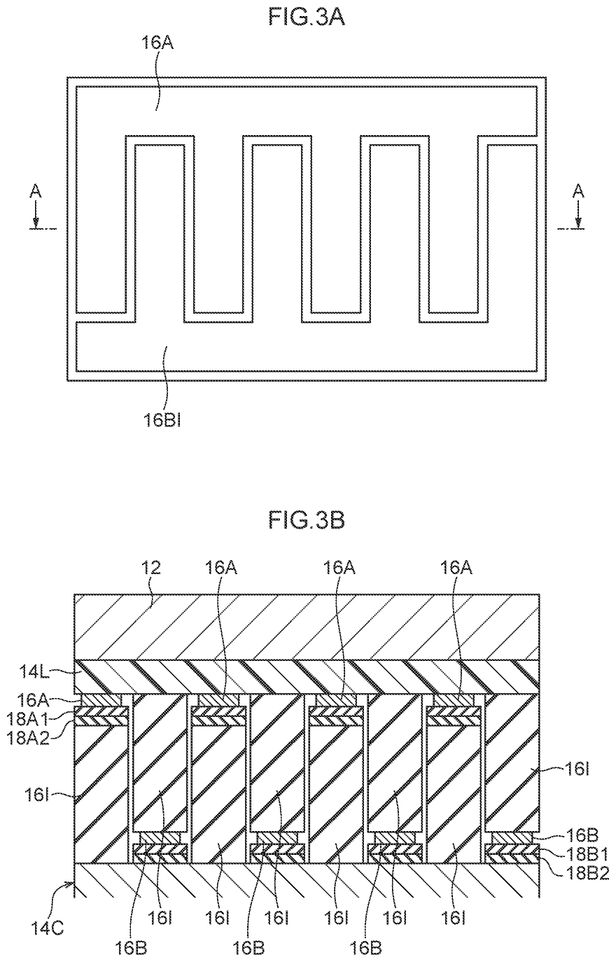

FIG. 3A is an enlarged drawing of the arranged pattern of the steering wheel electrodes 16A, 16B illustrated in FIG. 2, and FIG. 3B is a cross-sectional view at line A-A of FIG. 3A. As illustrated in FIG. 3B, the steering wheel electrodes 16A, 16B are respectively disposed on a core material 14C, which is made of resin or the like, of the steering wheel 14, and the steering wheel electrodes 16A, 16B are covered by a covering material 14L of leather or the like that structures the surface of the steering wheel 14. As illustrated in FIG. 3B, the steering wheel electrodes 16A, 16B respectively do not contact the body 12 directly, and are grasped by the vehicle occupant via the covering material 14L.

The steering wheel electrode 16A is disposed on the core material 14C via an insulating material 161 that has a predetermined thickness, and buffer materials 18A1, 18A2. The steering wheel electrode 16B is disposed on the core material 14C via buffer materials 18B1, 18B2. The insulating material 161 is disposed on the steering wheel electrode 16B that is disposed on the buffer materials 18B1, 18B2.

As illustrated in FIG. 3B, because the distance of the steering wheel electrode 16B from the hands (the body 12) of the vehicle occupant is greater than that of the steering wheel electrode 16A, the charge that is generated between the hand of the vehicle occupant and the steering wheel electrode 16B is lower than that of the steering wheel electrode 16A. As a result, the electrocardiographic signal detected at the steering wheel electrode 16B is smaller than the electrocardiographic signal detected at the steering wheel electrode 16A.

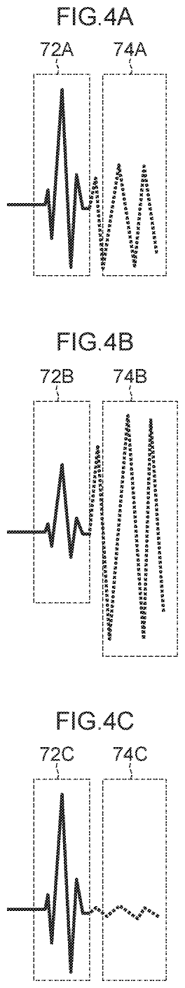

FIG. 4A is a schematic drawing illustrating an example of the electrocardiographic signal that is detected at the steering wheel electrode 16A and outputted from the waveform generator 40A. FIG. 4B is a schematic drawing illustrating an example of the electrocardiographic signal that is detected at the steering wheel electrode 16B and outputted from the waveform generator 40B. FIG. 4C is a schematic drawing illustrating an example of the electrocardiographic signal that is outputted from the signal processing section 60.

An R wave 72A is seen markedly in the electrocardiographic signal that is detected at the steering wheel electrode 16A and illustrated in FIG. 4A, and normal mode noise 74A that is due to vibration of the steering wheel electrodes 16A, 16B is suppressed relatively, and the S/N ratio of the signal is high. In the electrocardiographic signal that is detected at the steering wheel electrode 16B and illustrated in FIG. 4B, although an R wave 72B is seen, normal mode noise 74B that is due to vibration of the steering wheel electrodes 16A, 16B is marked, and as a result, the S/N ratio of the signal is low as compared with the electrocardiographic signal detected at the steering wheel electrode 16A.

In the present embodiment, the processing of outputting the difference between the electrocardiographic signal detected at the steering wheel electrode 16A and the signal, which is detected at the steering wheel electrode 16B and in which noise is marked, or the like is carried out by the signal processing section 60. Due to this processing, the component relating to the normal mode noise 74A is removed, and an electrocardiographic signal having a high S/N ratio is outputted.

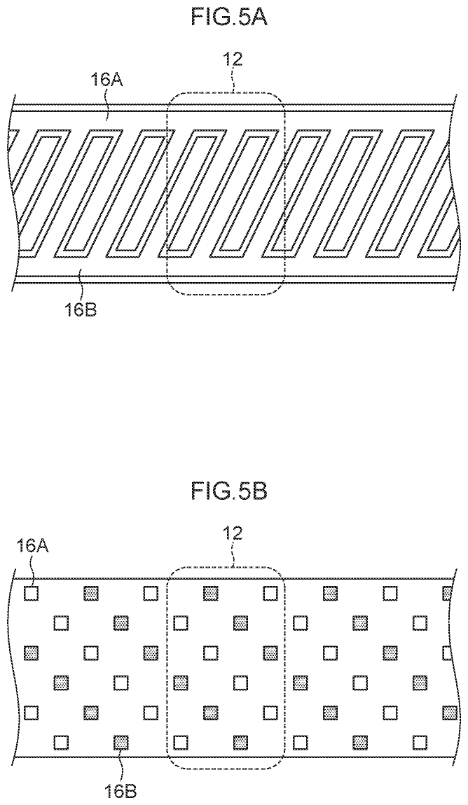

FIG. 5A is a schematic drawing illustrating a modified example of the arranged pattern of the steering wheel electrodes 16A, 16B, and FIG. 5B is a schematic drawing illustrating another example of a modified example of the arranged pattern of the steering wheel electrodes 16A, 16B. The steering wheel electrodes 16A, 16B illustrated in FIG. 5A are respectively disposed so as to be inclined with respect to the peripheral direction of the steering wheel 14. The steering wheel electrodes 16A, 16B illustrated in FIG. 5B are disposed in forms of dots.

In both the cases of FIG. 5A and FIG. 5B, as seen from the radial direction of the steering wheel 14, the steering wheel electrode 16A and the steering wheel electrode 16B respectively are symmetrical and are adjacent to one another, and the respective shapes thereof are substantially congruent, and the steering wheel electrodes 16A, 16B have a geometrically equivalent relationship in which the respective surface areas thereof are substantially the same. Further, the finer the arranged pattern of the steering wheel electrodes 16A, 16B, the higher the correlation with the noise that is detected.

In addition to the forms illustrated in FIG. 5A and FIG. 5B, arranged patterns of other forms may be used provided that the steering wheel electrode 16A and the steering wheel electrode 16B respectively have a geometrically equivalent relationship in the plane that is parallel to the surface of the steering wheel 14.

As described above, in accordance with the present embodiment, due to the steering wheel electrode 16B being placed further away from the hands of the vehicle occupant than the steering wheel electrode 16A, the normal mode noise 74B that enters from the non-grasped portion of the steering wheel electrode 16B is marked, and the R wave 72B that is obtained from the steering wheel electrode 16B is smaller than the R wave 72A that is obtained from the steering wheel electrode 16A. As a result, the electrocardiographic signal detected at the steering wheel electrode 16B has a lower S/N ratio than the electrocardiographic signal detected at the steering wheel electrode 16A. In the present embodiment, an electrocardiographic signal having a high S/N ratio can be obtained by the processing of outputting the difference between the electrocardiographic signal detected at the steering wheel electrode 16A and the signal, which is detected at the steering wheel electrode 16B and in which noise due to vibration is marked, or the like.

Note that the first electrode portion in the claims corresponds to the steering wheel electrode 16A in the present embodiment, the second electrode portion therein corresponds to the steering wheel electrode 16B in the present embodiment, the first detecting portion therein corresponds to the waveform generator 40A in the present embodiment, the second detecting portion therein corresponds to the waveform generator 40B in the present embodiment, and the signal processing section therein corresponds to the signal processing section 60 in the present embodiment.

### Second Embodiment

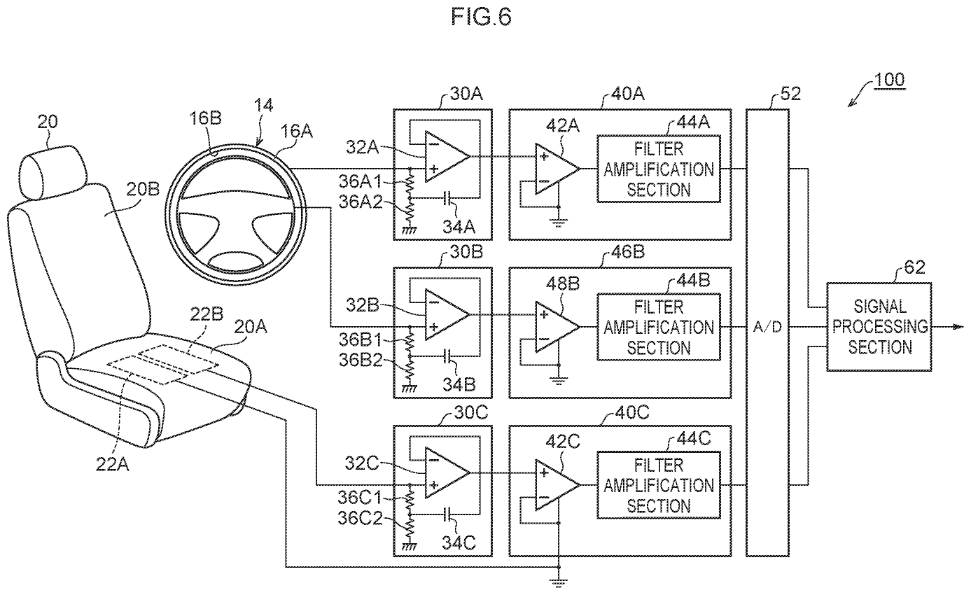

An electrocardiographic detection device 100 for a vehicle relating to a second embodiment is described next. FIG. 6 is a drawing illustrating the schematic structure of the electrocardiographic detection device 100 for a vehicle relating to the second embodiment. The electrocardiographic detection device 100 for a vehicle relating to the present embodiment differs from the electrocardiographic detection device 10 for a vehicle relating to the first embodiment with regard to the points of additionally having a seat electrode 22B, a buffer circuit 30C and a waveform generator 40C, and that an A/D converter 52 converts the analog signals outputted from the waveform generators 40A, 46B, 40C respectively into digital signals, and a signal processing section 62 generates an electrocardiographic signal of a high S/N ratio by using the signals outputted from the waveform generators 40A, 46B, 40C respectively, and the seat electrode 22A is grounded together with the inverting input terminal (−) of a differential amplifier 42C of the waveform generator 40C. Because the other structures are the same as those of the electrocardiographic detection device 10 for a vehicle relating to the first embodiment, the structures that are the same as the first embodiment are denoted by the same reference numerals, and detailed description thereof is omitted.

In the present embodiment, the steering wheel electrodes 16A, 16B, the buffer circuits 30A, 30B and the waveform generator 40A are the same as in the first embodiment. At the waveform generator 46B relating to the present embodiment, the filter amplification section 44B is the same as in the first embodiment, but a differential amplifier 48B is different from the differential amplifier 42B of the first embodiment with regard to the point that the seat electrode 22A is not connected to the inverting input terminal (−) of the differential amplifier 48B and is grounded.

The buffer circuit 30C is provided between the seat electrodes 22A, 22B and the waveform generator 40C, and outputs the signals from the seat electrodes 22A, 22B to the waveform generator 40C as a buffer. In the same way as the buffer circuit 30A, the buffer circuit 30C is structured by a positive feedback circuit that includes an operational amplifier 32C, resistors 36C1, 36C2, and a capacitor 34C.

The waveform generator 40C is structured to include the differential amplifier 42C that serves as a detecting portion, and a filter amplification section 44C, and, on the basis of the current signal inputted from the seat electrode 22B, generates an electrocardiographic waveform of the vehicle occupant that includes the common mode noise 130.

Due to the non-inverting input terminal (+) of the differential amplifier 42C being connected to the seat electrode 22B via the buffer circuit 30C, and the inverting input terminal (−) being grounded together with the seat electrode 22A, the differential voltage (differential signal) between the signal inputted to the non-inverting input terminal (+) and the signal inputted to the inverting input terminal (−) is detected, and is outputted to the filter amplification section 44C. Specifically, the differential amplifier 42B amplifies, by a predetermined factor, the difference between the input signal from the seat electrode 22B and GND, and outputs a signal.

The filter amplification section 44C amplifies the output signal of the differential amplifier 42C, and, in the same way as the above-described filter amplification section 44A, carries out the processing of converting the resultant signal into a signal of a frequency of a predetermined range by using a predetermined filter, or the like, and outputs the results of processing via the A/D converter 52 to the signal processing section 62.

The signal processing section 62 is structured by a computer including a CPU (Central Processing Unit), a ROM (Read Only Memory), a RAM (Random Access Memory), and a non-volatile memory such as a flash memory or the like. The CPU executes signal processing by executing a program that is stored in advance in the memory.

In the present embodiment, the processing of outputting the difference between a signal, in which the waveform of the electrocardiographic signal outputted from the waveform generator 40A is marked, and a signal, in which the common mode noise 130 and the normal mode noise 132 such as illustrated in FIG. 8 are markedly included in the electrocardiographic signal outputted from the waveform generator 46B, and a signal, in which the common mode noise 130 is markedly included in the electrocardiographic signal outputted from the waveform generator 40C, or the like, is carried out. Due to this processing, the components relating to the common mode noise 130 and the normal mode noise 132 are removed from the electrocardiographic signal outputted from the waveform generator 40A, and an electrocardiographic signal having a high S/N ratio is outputted.

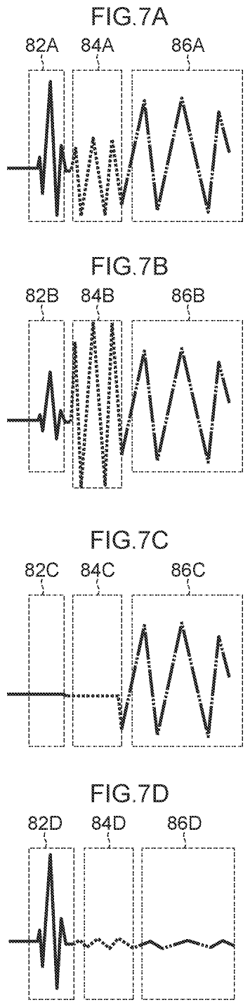

FIG. 7A is a schematic drawing illustrating an example of the electrocardiographic signal that is detected at the steering wheel electrode 16A and outputted from the waveform generator 40A. FIG. 7B is a schematic drawing illustrating an example of the electrocardiographic signal that is detected at the steering wheel electrode 16B and outputted from the waveform generator 46B. FIG. 7C is a schematic drawing illustrating an example of the electrocardiographic signal that is detected at the seat electrode 22B and outputted from the waveform generator 40C. FIG. 7D is a schematic drawing illustrating an example of the electrocardiographic signal that is outputted from the signal processing section 62.

An R wave 82A is seen markedly in the electrocardiographic signal that is detected at the steering wheel electrode 16A and illustrated in FIG. 7A, and normal mode noise 84A that is due to vibration of the steering wheel electrodes 16A, 16B is suppressed relatively. However, common mode noise 86A that is due to charges generated at the body 12 side is seen, and as a result, the S/N ratio of the signal is lowered. In the electrocardiographic signal that is detected at the steering wheel electrode 16B and illustrated in FIG. 7B, although an R wave 82B is seen, normal mode noise 84B that is due to vibration of the steering wheel electrodes 16A, 16B is marked, and moreover, common mode noise 86B that is due to charges generated at the body 12 side is seen. An R wave 82C and normal mode noise 84C that is due to vibration of the steering wheel electrodes 16A, 16B are not seen in the electrocardiographic signal that is detected at the seat electrode 22B and illustrated in FIG. 7C. However, common mode noise 86C that is due to charges generated at the body 12 side is seen.

In the present embodiment, the processing of outputting the difference between the electrocardiographic signal detected at the steering wheel electrode 16A, the signal detected at the steering wheel electrode 16B, and the signal detected at the seat electrode 22B, or the like is carried out at the signal processing section 62. Due to this processing, the components relating to the normal mode noise 84A and the common mode noise 86A are removed, and an electrocardiographic signal having a high S/N ratio is outputted.

As described above, in the present embodiment, an electrocardiographic signal of a high S/N ratio can be obtained by the processing of outputting the difference between the electrocardiographic signal detected at the steering wheel electrode 16A, the signal detected at the steering wheel electrode 16B and in which the normal mode noise 84B is seen markedly, and the signal detected at the seat electrode 22B and in which the common mode noise 86C is seen markedly, or the like.

Although the present embodiment describes a form in which electrocardiographic waveforms are detected by using the steering w % heel electrodes 16A, 16B and the seat electrodes 22A, 22B, the present invention is not limited to this. For example, a pair of seat electrodes that are provided at the seatback 20B may be used instead of the seat electrodes 22A, 22B.

Note that the first electrode portion in the claims corresponds to the steering wheel electrode 16A in the present embodiment, the second electrode portion therein corresponds to the steering wheel electrode 16B in the present embodiment, the third electrode portion therein corresponds to the seat electrode 22B in the present embodiment, the first detecting portion therein corresponds to the waveform generator 40A in the present embodiment, the second detecting portion therein corresponds to the waveform generator 46B in the present embodiment, the third detecting portion therein corresponds to the waveform generator 40C in the present embodiment, and the signal processing section therein corresponds to the signal processing section 62 in the present embodiment.

The processings carried out at the signal processing sections 60, 62 in the above-described embodiments may be software processings, or may be processings carried out by hardware. Or, these processings may be processings that combine both hardware and software.

Further, the processings carried out at the signal processing sections 60, 62 in the above-described embodiments may be stored as programs on storage media and distributed.

Moreover, the present invention is not limited to the above, and, other than the above, can of course be implemented by being modified in various ways within a scope that does not depart from the gist thereof.

The disclosure of Japanese Patent Application No. 2020-055102 filed on Mar. 25, 2020 is, in its entirety, incorporated by reference into the present specification.

1. An electrocardiographic detection device for a vehicle, comprising:
a first electrode portion provided at a lower layer of a covering material that covers a surface of a steering wheel;
a second electrode portion provided at the lower layer of the covering material of the steering wheel, an insulating material of a predetermined thickness being between the second electrode and the lower laver of the covering material;
a first detecting portion detecting a differential voltage between the first electrode portion and a ground region;
a second detecting portion detecting a differential voltage between the second electrode portion and the ground region; and
a signal processing section that generates an electrocardiographic signal based on the differential voltage detected at the first detecting portion and the differential voltage detected at the second detecting portion.

2. An electrocardiographic detection device for a vehicle, comprising:
a first electrode portion provided at a lower layer of a covering material that covers a surface of a steering wheel;
a second electrode portion provided at the lower layer of the covering material of the steering wheel, an insulating material of a predetermined thickness being between the second electrode and the lower laver of the covering material;
a third electrode portion provided at a seat in which a vehicle occupant sits;
a first detecting portion detecting a differential voltage between the first electrode portion and a ground region;
a second detecting portion detecting a differential voltage between the second electrode portion and a ground region;
a third detecting portion detecting a differential voltage between the third electrode portion and a ground region; and
a signal processing section that generates an electrocardiographic signal based on the differential voltage detected at the first detecting portion, the differential voltage detected at the second detecting portion, and the differential voltage detected at the third detecting portion.

3. The electrocardiographic detection device for a vehicle of claim 1, wherein the first electrode portion and the second electrode portion respectively are disposed so as to be symmetrical as seen from a radial direction of the steering wheel.

4. The electrocardiographic detection device for a vehicle of claim 3, wherein, as seen from the radial direction of the steering wheel, the first electrode portion and the second electrode portion respectively are adjacent to one another, respective shapes thereof are substantially congruent, and the first electrode portion and the second electrode portion have a geometrically equivalent relationship in which respective surface areas thereof are substantially the same.

5. The electrocardiographic detection device for a vehicle of claim 1, wherein the first electrode portion and the second electrode portion respectively are disposed parallel as seen from a radial direction of the steering wheel.

6. The electrocardiographic detection device for a vehicle of claim 1, wherein the first electrode portion and the second electrode portion are disposed in forms of dots as seen from a radial direction of the steering wheel.
